(19) United States
(12) Patent Application Publication
(10) Pub. No.: US 2026/0007432 A1
(43) Pub. Date: Jan. 8, 2026

(54) Spinal Fixing Rod with Custom Variable Mechanical Properties for Vertebral Column Surgery and Methods for its Design and Fabrication

(71) Applicants: Juan Carlos Rodriguez Olaverri, New York, NY (US); javier Pereiro de Lamo, Madrid (ES); Javier Van Baumberghen Teran, Torrejon del Rey (ES); Benito del Rio Lopez, Guadalix de la Sierra (ES)

(72) Inventors: Juan Carlos Rodriguez Olaverri, New York, NY (US); javier Pereiro de Lamo, Madrid (ES); Javier Van Baumberghen Teran, Torrejon del Rey (ES); Benito del Rio Lopez, Guadalix de la Sierra (ES)

(21) Appl. No.: 19/251,273

(22) Filed: Jun. 26, 2025

Related U.S. Application Data
(60) Provisional application No. 63/668,164, filed on Jul. 6, 2024.

(51) Int. Cl. A61B 17/70; A61B 34/10

(52) U.S. Cl. CPC A61B 17/7004; A61B 34/10; A61B 2034/105; A61B 2034/108

(57) ABSTRACT

This document pertains to a provisional patent application for an innovative spinal fixing rod designed for vertebral column surgery. The invention specifically addresses the need for spinal fixing rods with variable mechanical properties to enhance surgical fixation and, where needed, correction of vertebrae and their definitive surgical fixation. This document encompasses methods for designing and fabricating these variable mechanical properties spinal fixing rods, ensuring they meet the precise mechanical and anatomical requirements of individual patients. This document outlines the various embodiments, applications, and procedures related to the utilization and production of custom and off-the-shelf spinal fixing rods with variable mechanical properties.

This application claims the benefit of U.S. Provisional Patent Application No. 63/668,164, titled “Spinal Fixing Rods with Custom Variable Mechanical Properties for Vertebral Column Surgery and Methods for Their Design and Fabrication”, filed on Jul. 6, 2024, under 35 U.S.C. § 119 (e). The entire contents of the aforementioned provisional application are incorporated herein by reference.

### BACKGROUND OF THE INVENTION

The mechanical properties of a spinal fixing rod can be likened to those of a structural beam in engineering. Both elements are designed to bear loads, resist stresses, and maintain structural integrity under varying conditions. The principles of material selection, beam design and dimensions are critical to ensure optimal performance and durability of a beam under real load application, stress distribution, bending and torsional moments.

“When designing a beam, you start with the applied loads, beam length, allowable stresses (and sometimes allowable deflection), and you must determine what size beam to use.” (Pag, 102, Opus Citation: Barry Dupen (2016). Applied Strength of Materials for Engineering Technology. 10 ed. Purdue University).

Spinal fixing rod design for the surgical treatment of spinal pathologies should follow the same principles as the design of beams in structural engineering, while being restricted by the biocompatibility of the material(s) used in the rod and the physical constraints imposed by human anatomy, structures, and organs. The surgeon must consider not only the contour, diameter, and material composition of the spinal fixing rod but ideally also anticipate the required mechanical loads and stresses that the spinal fixing rod will sustain not only during the surgical procedure but throughout its entire expected lifetime when implanted in a particular patient.

Most spinal fixing rods used in spine surgery to address a broad range of vertebral column pathologies are designed with a cylindrical shape (circular transverse cross-section). This cylindrical design is convenient for both ease of fabrication and intraoperative shaping to the desired contour of the patient's spine. The use of spinal fixing rods with a circular transverse cross-section (perpendicular to its major length) also facilitates the insertion and definitive fixation of the spinal fixing rod into the proximal end housing of various vertebral fixing elements attached to one or more of the vertebrae of the segment or segments of the spine being surgically fixed, like the spinal fixing rod and proximal end housing tulip of the vertebral fixing element (pedicle screw) shown in FIG. 1.

Commonly used spinal fixing rods in spine surgery are made of resistant alloy materials such as high-grade stainless steel, chrome-cobalt, and titanium alloys. However, other spinal fixing rods have been designed using polyether-ether-ketone (PEEK), carbon fiber, and various other mechanically resistant and biocompatible elements or composites. The spinal fixing rods described in this invention can be made from any of these materials or any other biocompatible and mechanically resistant material.

Traditionally, spinal fixing rods have been provided in a straight longitudinal shape, to be cut to length and shaped intraoperatively during the surgical procedure. In recent years, however, there has been a surge in the use of patient-specific custom-shaped spinal fixing rods, pre-shaped before surgery according to a surgical plan made by the surgeon in advance. These custom-shaped spinal fixing rods have greatly facilitated work in the surgical theater by eliminating the cumbersome process of manually cutting and intraoperatively conforming the spinal fixing rods to the specific anatomy of the patient or the desired corrected anatomy of a deformed patient's spine.

As an alternative to these custom-shaped spinal fixing rods, some manufacturers and providers offer surgeons the option to choose from a predetermined range of already pre-bent spinal fixing rods. These off-the-shelf pre-bent spinal fixing rods come in various degrees of bending, lengths, and shapes.

Planning a spine surgical procedure that requires one or more spinal fixing rods and shaping these rods before surgery has the added advantage of increasing their mechanical resistance to fatigue and breakage. This process avoids the potential notches that can be made if the surgeon has to bend the rods manually or with automated benders in the surgical theater. These custom-shaped rods can even undergo industrial heat treatment after being precisely molded to the shape required by the surgeon for a particular patient, improving the molecular structure of the alloy and enhancing its resistance to breaking and fatigue.

Most spinal fixing rods currently in use are cylindrical, having a circular transverse cross-section. Others, like the “Mesa Rod” (K2M Inc, Leesburg, VA 20175), have a shape that combines a circular and an H-beam cross-section. However, all these spinal fixing rods—whether they have a circular transverse cross-section, a combination of a transverse cross-section and an H-beam, or any other type of transverse cross-section—share the characteristic of having a uniform transverse cross-section area and shape throughout their entire length.

Some more daring designs for a spinal fixation rod have been envisioned, offering the possibility of employing a spinal fixing rod in the form of a slender truncated cone, where the rod's transverse circular cross-section continuously diminishes in size from one terminal end to the other.

For clarity, throughout the description of this invention, the primary axis running along the major length of the spinal fixing rod will be designated as the z-axis. The x and y axes are orthogonal to the z-axis. An exemplary description of these axes in a particular embodiment of a spinal fixing rod with variable mechanical properties is shown in FIG. 2A. This figure depicts a cross-sectional view of the spinal fixing rod with variable mechanical properties along its entire length in the yz plane. Enlarged images of the transverse cross-section (xy plane) at three different points along the rod's length are illustrated in FIGS. 2B to 2D, highlighting the referenced axes.

After having defined the axes of for the exemplary spinal fixing rod of FIGS. 2A to 2D, any reference made in the following text to the transverse cross-section shape or area of a spinal fixing rod(s), unless otherwise specified, will refer to the cross-section shape or area of the spinal fixing rod(s) as seen in the xy plane define by the x and y axes described previously.

A cylindrical shape spinal fixing rod having a uniform circular transverse cross-section perpendicular to its major length, like those commonly used in today's spinal surgery, presents potential disadvantages. These spinal fixing rods are made of isotropic materials, providing the rod with uniform mechanical properties along the x, y, and z axes.

But the common use of spinal fixing rods with a uniform transverse circular cross-section made of isotropic materials presents some mechanical disadvantages. In some patients, the mechanical strength and flexural rigidity provided by these spinal fixing rods are insufficient to bear the stresses and strains they must endure, leading to failure and breakage. In other patients, the flexural rigidity of the implanted spinal fixing rods is too high at the proximal end (the most cranial part) of the vertebral column being fixed, contributing to the development of proximal junctional kyphosis and proximal junctional failure. These latter conditions can occur when one or more vertebrae above the surgically fixed segment of the spine are displaced into kyphosis or fracture, leading to pain and deterioration in the patient's health status. Failure and breakage of the spinal fixing rods, as well as the development of proximal junctional kyphosis or failure in the superior adjacent level of the spine that has been surgically fixed, can require additional surgical interventions to address these issues.

While increasing the cross-sectional diameter and area of a circular transverse cross-section spinal fixing rod reduces the chances of spinal fixing rod failure due to mechanical fatigue and breakage, this increase in surface area can be detrimental to the segment of the spine at the most cranial end of the vertebral column being surgically fixed. A larger diameter spinal fixing rod will significantly increase the flexural rigidity of the implanted construct, thereby increasing the risk of proximal junctional kyphosis and failure. Conversely, reducing the diameter of a circular transverse cross-section spinal fixing rod can mitigate the development of proximal junctional kyphosis and failure in the patient. However, this reduction in diameter and area inherently increases the risk of rod breakage and failure in other areas of the spinal fixation where the mechanical demands are greater.

To address these conflicting problems, some spinal fixing rods with a circular transverse cross-section have been designed with two or more clearly differentiated diameters along their length, maintaining the same transverse cross-section shape, except for the diameter and area. These spinal fixing rods provide different flexural rigidity to various segments of the vertebral column being fixed. The proximal (most cranial) end and segments of the spinal fixing rod have smaller diameters to reduce the chances of proximal junctional kyphosis and failure, while the most caudal segments and end have greater diameters to increase flexural rigidity and mechanical strength, thereby reducing the likelihood of rod breakage under physiological loads. However, these spinal fixing rods, which feature two or more clearly differentiated diameters of the same circular transverse cross-section shape and, therefore, different transverse cross-sectional areas along their length, must also be used with vertebral fixing elements, such as pedicle screws, that must have different sizes at their proximal end housing to accommodate, hold, and firmly fix the varying diameters of the spinal fixing rods. This requirement imposes additional burdens on the selection of the vertebral fixing elements to be linked and fixed to the spinal fixing rod.

### SUMMARY OF THE INVENTION

This invention addresses two significant disadvantages of conventional spinal fixing rods used in the surgical treatment of vertebral column pathologies that require the correction and/or fixation of one or more vertebral segments: rod breakage and proximal junctional failure resulting in kyphosis. The invention introduces a variable mechanical properties (VMP) spinal fixing rod designed to offer adjustable mechanical resistance along its length, with increased rigidity where necessary and reduced flexural rigidity at the proximal (most cranial) end to minimize junctional failure and kyphosis. Surgeons can specify these mechanical requirements through detailed pre-surgical planning or select from pre-determined mechanical properties of off-the-shelf VMP spinal fixing rods.

A novel VMP spinal fixing rod is described that specifically addresses the individual mechanical requirements of a patient needing surgical intervention of the spine, where two or more vertebrae of the vertebral column require surgical fixation or correction and subsequent fixation. The spinal fixation using the VMP spinal fixing rods of this invention can extend to part or the entirety of the spine and may also include the cranium, sacrum, or pelvis. For simplicity, all such types of fixations will be designated as “spinal fixation” throughout this text.

The VMP spinal fixing rod of this invention can have any initial transverse cross-sectional shape, including but not limited to circular, ellipsoidal, polygonal, or other geometrical shapes, provided that these shapes allow the rod to be securely attached to vertebral fixing elements positioned on one or more vertebrae of the spinal column. This flexibility in shape is intended to ensure compatibility with a wide range of vertebral fixing elements used in spinal surgeries. The transverse cross-section of the VMP spinal fixing rod can also feature combinations of these shapes to form more complex profiles. Additionally, the VMP spinal fixing rod of this invention may be partially or completely hollowed along one or more segments of its major length if so desired, adding further complexity and variety to the possible configurations. The specific geometry and maximum dimensions along the x and y axes of the transverse cross-section will be referred to as the “transverse initial cross-section” in the context of this invention or simply the “initial cross-section.” Examples of some initial cross-sections of these VMP spinal fixing rods are illustrated in FIGS. 3A to 3X. This initial cross-section shape, along with the material composition of the VMP spinal fixing rod, will determine the maximum mechanical resistance properties of the VMP spinal fixing rod. It is understood that various forms of initial cross-section shapes are included within the scope of this invention, as long as they meet the functional requirement of attachment to vertebral fixing elements.

The VMP spinal fixing rod of this invention has an initial transverse cross-section shape that can vary along the major length (z-axis) of the VMP spinal fixing rod, either in one or more segments or along its entire length, providing different mechanical properties along the major length of the VMP spinal fixing rod.

In a preferred embodiment, the change in the initial transverse cross-section shape of the VMP spinal fixing rod occurs by continuously diminishing the height (y-axis) of this initial cross-section at one or more segments along the major length (z-axis) of the VMP spinal fixing rod. A particular embodiment of a VMP spinal fixing rod, where the height (y-axis) of the transverse cross-section diminishes along a significant length of the rod, is depicted in a perspective view in FIG. 4.

In a preferred embodiment, the initial transverse cross-section shape of the VMP spinal fixing rod will be situated at the distal (most caudal) end of the VMP spinal fixing rod and can be maintained along a longer or shorter length at this distal end. However, in an alternative embodiment, the maximum height (y-axis) of the transverse cross-section, and therefore its initial transverse cross-section shape, will not be situated at the distal end and can be placed anywhere along the major length (z-axis) of the VMP spinal fixing rod. Additionally, this maximum height (y-axis) of the transverse cross-section can be placed in two or more different segments along its major length (z-axis).

The change in the height (y-axis) of the VMP spinal fixing rod, along one or more segments or the entire length of the rod, will be, in one preferred embodiment, continuous and smooth, ensuring a seamless transition without abrupt changes. This is reflected in a cross-section (yz plane) of the VMP spinal fixing rods seen in FIGS. 5A to 5D. However, many other forms of continuous and smooth changes in the height of the transverse cross-section can be devised if desired. In alternative embodiments, more abrupt, angular, or discrete changes in the height of the transverse cross-section can be implemented in the design of the VMP spinal fixing rod if needed. Some examples of these VMP spinal fixing rods, with more discrete or abrupt changes in their height (y-axis), as seen in the yz plane, are shown in FIGS. 6A and 6C.

As seen in the different embodiments depicted in FIGS. 4, 5A to 5D and 6A to 6C, the height (y-axis) of the VMP spinal fixing rod can be maintained, increased or even decreased along one or more segments of the length (z-axis) of the rod.

In a preferred embodiment, the three-dimensional shape along the major length (z-axis) of the VMP spinal fixing rods disclosed in this invention will be planned before surgery for use in a particular patient's surgical procedure. These custom-shaped VMP spinal fixing rods will be adapted to be fixed onto the vertebral column of the patient or onto the vertebral column after the patient's spinal deformity has been corrected.

In this preferred embodiment, these three-dimensional contoured VMP spinal fixing rods will be molded prior to surgery and are commonly referred to as custom-made rods. However, since there are already VMP spinal fixing rods in use that are shaped to the contour of a patient's spine following a preoperative surgical plan, in the context of our patent description, these other VMP spinal fixing rods will be referred to as “custom-shaped VMP spinal fixing rods,” as they are customized to shape mostly the three-dimensional contour of the patient's spine.

These existing custom-shaped rods, designed according to a preoperative surgical plan, are to be distinguished from the “custom-made VMP spinal fixing rods” of our invention, which are tailored not only to the anatomical and desired three-dimensional shape of the patient's spine but also to their mechanical needs.

In a preferred embodiment, these VMP spinal fixing rods with variable height (y-axis), as described herein, are designed to be easily inserted and accommodated in the dedicated proximal housing elements of vertebral fixing elements attached to one or more vertebrae of the patient's spine. Particular examples of possible couplings between VMP spinal fixing rods and corresponding vertebral fixing elements can be seen in FIGS. 7A to 71. As shown in the exemplary embodiments of FIGS. 7A to 71, the housing elements of the vertebral fixing elements closely accommodate the constant, invariable inferior part of the shape of the transverse cross-section of the VMP spinal fixing rod.

In another alternative embodiment, the change in the initial transverse cross-section shape occurs in one or more segments along the length of the VMP spinal fixing rod, or even along its entire length, by continuously diminishing the width (x-axis) of the transverse cross-section while maintaining the height (y-axis) of this transverse cross-section. The decrement in the width of the VMP spinal fixing rod will be continuous and linear in one preferred embodiment, however, other forms of continuous and non-linear decrements in the width (x-axis) of the transverse cross-section along the VMP spinal fixing rod can be devised if desired. In alternative embodiments, more abrupt, steeper, or even discontinuous decrements in the width of the transverse cross-section can be implemented in the design of the VMP spinal fixing rod if desired. Some examples of the transverse cross-section of these VMP spinal fixing rods, where the width (x-axis) varies while the height (y-axis) remains constant, are shown in FIG. 8A to 8D.

In the embodiments of FIGS. 7A to 7I, the height (y-axis) of the VMP spinal fixing rod may be reduced significantly along one or more segments or the entire length of the rod, and this reduction can occur to the extent that the width of the rod is also diminished if the closing nut of the vertebral element is designed to allow such a reduction. For VMP spinal fixing rods that are inserted laterally, like those shown in FIGS. 8A to 8D, where the width typically decreases, the width can be reduced so much that the height of the medial part of the rod starts to diminish. These variations ensure the adaptability of the VMP spinal fixing rods to different surgical requirements and anatomical constraints and are also considered within the scope of this invention.

In another alternative embodiment, the change in the initial transverse cross-section shape occurs in one or more segments along the length of the VMP spinal fixing rod, or even along its entire length, without altering the maximum width (x-axis) or height (y-axis) of the transverse cross-section. The change in the shape and area of the VMP spinal fixing rod will be continuous and linear in one preferred embodiment. However, other forms of continuous and non-linear changes in the shape and area of the transverse cross-section, while maintaining the maximum width (x-axis) and height (y-axis) along the major length of the VMP spinal fixing rod, can also be designed if desired, providing the VMP spinal fixing rod with different mechanical properties. In alternative embodiments, more abrupt, steeper, or even discontinuous changes in the shape and area of the transverse cross-section of the VMP spinal fixing rod, while maintaining the maximum width (x-axis) and height (y-axis) along its major length, can be implemented if desired. Two particular examples of VMP spinal fixing rods with changes in the transverse cross-section area and shape that maintain the maximum width (x-axis) and height (y-axis) along the length of the rod are shown in FIG. 9. In FIG. 9, the transverse cross-sections of these two VMP spinal fixing rods are compared with those of a normal circular transverse cross-section spinal fixing rod at different segments along the length of the rods.

Embodiments of these VMP spinal fixing rods with variable width (x-axis), as described herein, are designed to be inserted and accommodated in the dedicated proximal housing elements of one or more vertebral fixing elements attached to the patient's spine. These vertebral fixing elements must have the required height and shape to accommodate the constant part of the transverse cross-section shape (xy plane) of the VMP spinal fixing rod, allowing for “lateral” or “medial” insertion, as opposed to the more common “superior” or “posterior” insertion of most VMP spinal fixing rods employed today. Specific embodiments of these vertebral fixing elements with lateral openings for the insertion and fixation of these VMP spinal fixing rods are illustrated in FIGS. 8A to 8D.

The changes in the shape of the transverse cross-section (xy plane), as described above, are essential to provide the desired differentiated mechanical properties to the VMP spinal fixing rod of this invention. By varying its transverse cross-section (xy plane), the VMP spinal fixing rod can offer variable or differentiated mechanical properties to the segment or segments of the spine being surgically fixed.

A combination of different methods for altering the mechanical properties can also be employed along the same rod. This includes changing the height (y-axis) while maintaining the width (x-axis), changing the width (x-axis) while maintaining the height (y-axis), and changing the shape without altering either the maximum height or width. By mixing these approaches within the same rod, it is possible to customize the VMP spinal fixing rod to meet specific mechanical requirements and anatomical constraints of individual patients. This combination of techniques allows for greater flexibility in the design and functionality of VMP spinal fixing rods, ensuring they provide optimal support and correction as needed. These combined variations ensure the adaptability of the VMP spinal fixing rods to different surgical requirements and anatomical constraints and are also considered within the scope of this invention.

The variation in the initial transverse cross-section (xy plane) shape of the VMP spinal fixing rods, as described in this invention, is specifically designed prior to surgery to meet the mechanical demands of the segment(s) of the particular patient's spine being fixed or corrected and fixed. The goal of these pre-surgically determined changes to the transverse cross-section shape is to ensure that the VMP spinal fixing rod can adequately address the diverse mechanical stresses encountered in different regions of the patient's spine. This design aims to improve the overall effectiveness and durability of the spinal correction and/or fixation performed surgically with these VMP spinal fixing rods.

In a preferred embodiment, the mechanical requirements of the VMP spinal fixing rods are pre-established or selected by the surgeon during a surgical planning process facilitated by medical imaging and surgical planning software, such as the one described below. The preplanned mechanical requirements will ensure the VMP spinal fixing rods can sustain the loads, stresses, and bending moments expected during the correction of the malaligned spine and the insertion and fixation of the VMP spinal fixing rods onto vertebral fixing elements.

Additionally, in a preferred embodiment, the determination of the mechanical requirements for the VMP spinal fixing rod, as designed or selected during the surgical planning process, will also take into consideration the physiological forces that will occur along the patient's spine after surgery, throughout their lifetime and daily activities.

In a preferred embodiment, the mechanical requirements imposed on the VMP spinal fixing rod, as designed by the surgeon, will result in a rod with continuous, non-discrete changes in the shape of its transverse cross-section (xy plane), ensuring no significant discontinuities along its length (z-axis). However, in alternative embodiments, more discrete or abrupt changes in the shape of the transverse cross-section (xy plane) of the VMP spinal fixing rod can be implemented if desired by the surgeon. These alternative embodiments are also covered under the scope and protection of this invention.

As is now apparent, in a preferred embodiment of this invention, the surgeon will be able to specify both the final desired three-dimensional shape and the mechanical properties of the VMP spinal fixing rods to be used in a spinal surgical procedure of a particular patient, using an appropriate surgical planning process, as described later in this document.

In an alternative embodiment of this invention, the surgeon may opt to use “straight” VMP spinal fixing rods with varying mechanical properties along one or more segments or their entire length. These variations are achieved through changes in the transverse cross-section (xy plane) shape along the major length of the rods (z-axis), as described herein. In this embodiment, the manufacturer can provide the rods in a straight longitudinal shape, which can be cut to the desired length and contoured to the desired shape by the surgeon during the surgical procedure using manual or automated mechanisms. These straight rods, when made in a manner similar to that described herein to provide variable mechanical properties, are also included under the scope of this invention.

In other alternative embodiments, straight VMP spinal fixing rods with variable mechanical properties, determined and fixed by the provider through changes in the transverse cross-section (xy plane) shape as described herein, can be cut to length and bent to a desired shape following surgical preplanning. These VMP spinal fixing rods can then be sterilized for use and can be re-cut and re-contoured during the surgical procedure if necessary. These pre-bent and pre-sterilized VMP spinal fixing rods, made to provide variable mechanical properties, are also included under the scope of this patent. In a variation of this alternative particular embodiment, the provider, using a straight VMP spinal fixing rod, determined and fixed by the provider through changes in the transverse cross-section (xy plane) shape as described herein, can only cut to the desired length requested in the surgical planning done by the surgeon, leaving the contouring-shaping of the VMP spinal fixing rod and eventual re-cutting of the VMP spinal fixing rods to the surgeon, changes that can be made prior to surgery and/or during the surgical procedure. The employment of these rods, when designed in this way, is also included under the aim and scope of this invention.

In another alternative embodiment, the VMP spinal fixing rod can be provided with variable mechanical properties determined and fixed by the provider, achieved through changes in the transverse cross-section (xy plane) shape as described herein, and pre-bent into one or more off-the-shelf “standard” shapes. These rods are provided to the surgeon already packed and sterilized. These VMP spinal fixing rods can be surgically implanted onto the patient's spine after being reshaped or cut in the operating theater or before surgery, if desired. These uses of the VMP spinal fixing rods are included within the scope of this invention when these off-the-shelf variable mechanical properties and pre-bent rods are fabricated to provide differentiated mechanical properties along their major length by altering the shape of their transverse cross-section (xy plane), as described in this invention.

In one preferred embodiment, the VMP spinal fixing rod with variable mechanical properties along its major length, as described herein, will be over-contoured to not only meet a surgically planned desired final three-dimensional shape of the patient's spine but also to address the correction and/or fixation forces generated during surgical maneuvers. This includes the definitive fixation of the VMP spinal fixing rod onto the vertebral fixing elements that hold firmly to the vertebrae of one or more segments of the patient's spine.

In another preferred embodiment, the VMP spinal fixing rod with variable mechanical properties along its major length, as described herein, will be over-contoured to not only meet the desired final three-dimensional shape of the patient's spine and account for the correction and fixation forces generated during the surgical procedure but also to accommodate the expected physiological forces that the spine, along with the VMP spinal fixing rods, will endure post-surgery, such as those experienced when the patient is standing or walking. This over-contouring ensures that the VMP spinal fixing rod and the spine will attain their final desired shape once placed and firmly fixed onto the vertebral column of the patient.

In one preferred embodiment, the VMP spinal fixing rods of this invention will be over-contoured as described above to address the malaligned spine correctional forces, fixation forces, and physiological forces acting in one or more of the following planes, if so desired: sagittal, coronal, and axial.

A particular exemplary design of a VMP spinal fixing rod made of surgical titanium, with a maximum width of 5 mm and variable height to accommodate the expected spinal malalignment reduction and physiological forces, is depicted in FIG. 10. This VMP spinal fixing rod was designed following the desired “relative bending stiffnesses” along its length, compared to a standard cylindrical-shaped spinal fixing rod with a 5.5 mm diameter made of the same surgical titanium, which has a relative bending stiffness of 1.

The relative bending stiffness shown in FIGS. 10 to 13 refers to the bending stiffness of each spinal fixing rod along the imaginary x-axis passing through the centroid of the transverse cross-section of the rod, as defined previously in this invention. The expected spinal malalignment reduction and physiological forces are shown in the middle-central rods in FIGS. 10 to 13, where the forces are expressed in Newtons. Forces placed below the rod indicate strain forces applied to the rod, and forces above the rod represent support or compression reactive forces.

For all the spinal fixing rods depicted in FIGS. 10 to 13, the same length of 240 mm has been chosen, along with the same final expected three-dimensional shape of the rod as seen in the sagittal plane (yz-plane). Differences in the required pre-bending of the spinal fixing rods specified in FIGS. 10 to 13 can be observed for rods made of different materials with the same length and an equal expected final three-dimensional shape, under the action of the same expected spinal malalignment reduction and physiological forces in the sagittal plane (yz-plane).

FIG. 14 presents a comparison of the differences in the pre-bending needed for the spinal fixing rods shown in FIGS. 10 to 13 to achieve a similar final three-dimensional desired shape in the sagittal plane (yz-plane).

However, in an alternative embodiment using VMP spinal fixing rods as described in this invention, the surgeon has the flexibility to plan and design the over-contouring of the custom-made VMP spinal fixing rod by considering the forces that will act on the rod in as much detail as desired. The surgeon can choose to address the forces not only in the sagittal plane but also in one or more of the sagittal, coronal, and axial planes. This allows the surgeon to account for the forces exerted onto the VMP spinal fixing rod by the vertebral fixing elements attached to the vertebrae of the spinal fixation, ensuring an appropriate design and fixation of the VMP spinal fixing rod along the length of the spinal fixation.

### BRIEF DESCRIPTION OF THE DRAWINGS

FIG. 1: An image of a pedicle screw-type vertebral fixing element with commonly used cylindrical-shaped spinal fixing rods shown.

FIG. 2A: A diagrammatic representation of the reference x, y, z-axes on a VMP spinal fixing rod. The z-axis runs along the major length of the VMP spinal fixing rod.

FIG. 2B: A detailed view of the x and y-axes on the transverse cross-section at the proximal end of the rod.

FIG. 2C: A detailed view of the x and y-axes on the transverse cross-section at the mid-length of the rod.

FIG. 2D: A detailed view of the x and y-axes on the transverse cross-section at the distal end of the rod.

FIG. 3A to FIG. 3W: Various possible embodiments of initial cross-sections for the design and fabrication of a VMP spinal fixing rod.

FIG. 4: Perspective view of the particular embodiment of the straight VMP spinal fixing rod of FIG. 2a showing the overall shape along its major length.

FIG. 5A: A diagrammatic representation of a yz-plane cross-section of a first embodiment of a VMP spinal fixing rod showing continuous and smooth changes in the height (y-axis) along one or more segments.

FIG. 5B: A diagrammatic representation of a yz-plane cross-section of a second embodiment of a VMP spinal fixing rod showing continuous and smooth changes in the height (y-axis) along one or more segments.

FIG. 5C: A diagrammatic representation of a yz-plane cross-section of a third embodiment of a VMP spinal fixing rod showing continuous and smooth changes in the height (y-axis) along one or more segments.

FIG. 5D: A diagrammatic representation of a yz-plane cross-section of a third embodiment of a VMP spinal fixing rod showing continuous and smooth changes in the height (y-axis) along one or more segments.

FIG. 6A: A diagrammatic representation of a yz-plane cross-section of a first embodiment of a VMP spinal fixing rod showing non-linear changes in their height (y-axis)) along one or more segments of the length of the rod.

FIG. 6B: A diagrammatic representation of a yz-plane cross-section of a first embodiment of a VMP spinal fixing rod showing non-linear changes in their height (y-axis)) along one or more segments of the length of the rod.

FIG. 7A: A first alternative embodiment for coupling between a VMP spinal fixing rod and its corresponding vertebral fixing element, where the housing element of the vertebral fixing element closely accommodate the constant, invariable part of the transverse cross-section shape of the VMP spinal fixing rods. In this view of the transverse cross-section, the initial cross-section of the VMP with its maximum height (y-axis) can be observed.

FIG. 7B: A cross-section view of the same alternative embodiment for couplings between the VMP spinal fixing rod of FIG. 7A and its corresponding vertebral fixing element at a different segment along the major length of the VMP spinal fixing rod, where the maximum dimension of the width (x-axis) of the transverse cross-section remains constant while its height (y-axis) varies.

FIG. 7C: A cross-section view of the same alternative embodiment for couplings between the VMP spinal fixing rod of FIG. 7A and its corresponding vertebral fixing element at a more distant segment along the major length of the VMP spinal fixing rod, where the maximum dimension of the width (x-axis) of the transverse cross-section remains constant while its height (y-axis) has greatly diminished.

FIG. 7D: A second alternative embodiment for couplings between a VMP spinal fixing rod and its corresponding vertebral fixing element, similar to the embodiment shown in FIG. 7A. The initial cross-section of the VMP with its maximum height (y-axis) can be observed, but with a different design in the housing elements of the vertebral fixing elements.

FIG. 7E: A cross-section view of the same second alternative embodiment for couplings between the VMP spinal fixing rod of FIG. 7D and its corresponding vertebral fixing element at a different segment along the major length of the VMP spinal fixing rod, where the maximum dimension of the width (x-axis) of the transverse cross-section remains constant while its height (y-axis) varies.

FIG. 7F: A cross-section view of the same second alternative embodiment for couplings between the VMP spinal fixing rod of FIG. 7D and its corresponding vertebral fixing element at a more distant segment along the major length of the VMP spinal fixing rod, where the maximum dimension of the width (x-axis) of the transverse cross-section remains constant while its height (y-axis) has greatly diminished.

FIG. 7G: A third alternative embodiment for couplings between a VMP spinal fixing rod and its corresponding vertebral fixing element, similar to the embodiment shown in FIG. 7A and FIG. 7D. The initial cross-section of the VMP with its maximum height (y-axis) can be observed, but with a different design in the housing element of the vertebral fixing element.

FIG. 7H: A cross-section view of the same third alternative embodiment for couplings between the VMP spinal fixing rod of FIG. 7G and its corresponding vertebral fixing element at a different segment along the major length of the VMP spinal fixing rod, where the maximum dimension of the width (x-axis) of the transverse cross-section remains constant while its height (y-axis) varies.

FIG. 7I: A cross-section view of the same third alternative embodiment for couplings between the VMP spinal fixing rod of FIG. 7G and its corresponding vertebral fixing element at a more distant segment along the major length of the VMP spinal fixing rod, where the maximum dimension of the width (x-axis) of the transverse cross-section remains constant while its height (y-axis) has greatly diminished.

FIG. 8A: A first alternative embodiment for couplings between a VMP spinal fixing rod and its corresponding vertebral fixing element, where the housing element of the vertebral fixing elements closely accommodate the constant, invariable part of the transverse cross-section shape of the VMP spinal fixing rods. In this view of the transverse cross-section, the initial cross-section of the VMP with its maximum width (x-axis) can be observed.

FIG. 8B: A cross-section view of the same alternative embodiment for couplings between the VMP spinal fixing rod of FIG. 8A and its corresponding vertebral fixing element at a different segment along the major length of the VMP spinal fixing rod, where the maximum dimension of the height (y-axis) of the transverse cross-section remains constant while its width (x-axis) varies significantly.

FIG. 8C: A second alternative embodiment for couplings between a VMP spinal fixing rod and its corresponding vertebral fixing elements, similar to the embodiment shown in FIG. 8A. The initial cross-section of the VMP with its maximum width (x-axis) can be observed, but with a different design in the housing element of the vertebral fixing element.

FIG. 8D: A cross-section view of the same second alternative embodiment for couplings between the VMP spinal fixing rod of FIG. 8C and its corresponding vertebral fixing element at a more distant segment along the major length of the VMP spinal fixing rod, where the maximum dimension of the height (y-axis) of the transverse cross-section remains constant while its width (x-axis) has greatly diminished.

FIG. 8E: A third alternative embodiment for couplings between a VMP spinal fixing rod and its corresponding vertebral fixing elements, similar to the embodiment shown in FIG. 8A and FIG. 8C. The initial cross-section of the VMP with its maximum width (x-axis) can be observed, but with a different design in the housing element of the vertebral fixing element.

FIG. 8F: A cross-section view of the same second alternative embodiment for couplings between the VMP spinal fixing rod of FIG. 8C and its corresponding vertebral fixing element at a more distant segment along the major length of the VMP spinal fixing rod, where the maximum dimension of the height (y-axis) of the transverse cross-section remains constant while its width (x-axis) has greatly diminished.

FIGS. 9A to 9C: Cross-sectional views of a first alternative embodiment of VMP spinal fixing rod with changes in the transverse cross-section area and shape that maintain the maximum width (x-axis) and height (y-axis) along the entire length of the rod at different points along its length.

FIGS. 9D to 9F: Cross-sectional views of a second alternative embodiment of VMP spinal fixing rod with changes in the transverse cross-section area and shape that maintain the maximum width (x-axis) and height (y-axis) along the entire length of the rod at different points along its length.

FIG. 9G: A perspective view of the first alternative embodiment of the VMP spinal fixing rod corresponding to the cross-sectional views shown in FIGS. 9A to 9C.

FIG. 9H: A perspective view of the second alternative embodiment of the VMP spinal fixing rod corresponding to the cross-sectional views shown in FIGS. 9D to 9F.

FIG. 10A: A sagittal view of a particular embodiment of a custom-made titanium VMP spinal fixing rod having a length of 240 mm, a maximum width of 5.0 mm, and variable height along its entire length. This figure shows the final desired shape of the rod once implanted and subject to the expected forces.

FIG. 10B: The pre-bent custom-made titanium VMP spinal fixing rod of FIG. 10A with the required bending stiffness along the length of the rod. The bending stiffness refers to the bending stiffness across the x-axis that passes along the centroid of the transverse cross-section at different points along the rod, shown as a ratio compared to the bending stiffness of a 5.5 mm cylindrical-shaped commonly used spinal fixing rod made of the same titanium material.

FIG. 10C: The same pre-bent custom-made titanium VMP spinal fixing rod of FIG. 10B with the “expected” sagittal forces applied, expressed in Newtons. Forces below the rod are strain forces, and forces above the rod are support or compression forces.

FIG. 11A: A sagittal view of a 5.5 mm cylindrical-shaped Titanium rod having a length of 240 mm. This figure shows the final desired shape of the rod once implanted and subject to the expected forces, similar to the final desired shape in FIG. 10A.

FIG. 11B: The pre-bent 5.5 mm cylindrical-shaped Titanium rod of FIG. 11A showing its uniform relative bending stiffness along the length of the rod.

FIG. 11C: The same pre-bent 5.5 mm cylindrical-shaped Titanium rod of FIG. 11B with the same “expected” sagittal forces depicted in FIG. 10C, expressed in Newtons. Forces below the rod are strain forces, and forces above the rod are support or compression forces.

FIG. 12A: A sagittal view of a 5.5 mm cylindrical-shaped High Grade Stainless-Steel rod having a length of 240 mm. This figure shows the final desired shape of the rod once implanted and subject to the expected forces, similar to the final desired shape in FIG. 10A.

FIG. 12B: The pre-bent 5.5 mm cylindrical-shaped High Grade Stainless-Steel rod of FIG. 12A with its uniform relative bending stiffness along the length of the rod.

FIG. 12C: The same pre-bent 5.5 mm cylindrical-shaped High Grade Stainless-Steel rod of FIG. 12B with the same “expected” sagittal forces depicted in FIG. 10C, expressed in Newtons. Forces below the rod are strain forces, and forces above the rod are support or compression forces.

FIG. 13A: A sagittal view of a 5.5 mm cylindrical-shaped Chrome-Cobalt rod having a length of 240 mm. This figure shows the final desired shape of the rod once implanted and subject to the expected forces, similar to the final desired shape in FIG. 10A.

FIG. 13B: The pre-bent 5.5 mm cylindrical-shaped Chrome-Cobalt rod of FIG. 13A showing its uniform relative bending stiffness along the length of the rod.

FIG. 13C: The same pre-bent 5.5 mm cylindrical-shaped Chrome-Cobalt rod of FIG. 13B with the same “expected” sagittal forces depicted in FIG. 10C, expressed in Newtons. Forces below the rod are strain forces, and forces above the rod are support or compression forces.

FIG. 14A: A sagittal view of the pre-bent custom-made Titanium VMP spinal fixing rod from FIG. 10B, before the sagittal forces are applied.

FIG. 14B: A sagittal view of the pre-bent Titanium cylindrical shape spinal fixing rod from FIG. 11B, before the sagittal forces are applied.

FIG. 14C: A sagittal view of the pre-bent High-Grade Stainless-Steel cylindrical shape spinal fixing rod from FIG. 12B, before the sagittal forces are applied.

FIG. 14D: A sagittal view of the pre-bent Chrome-Cobalt cylindrical shape spinal fixing rod from FIG. 13B, before the sagittal forces are applied.

FIG. 15: Flowchart of Alternative Ways for the Use of VMP Spinal Fixing Rod

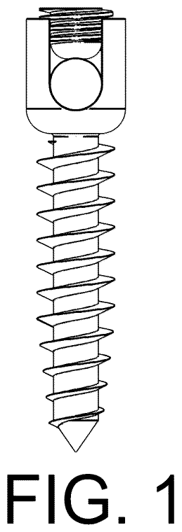

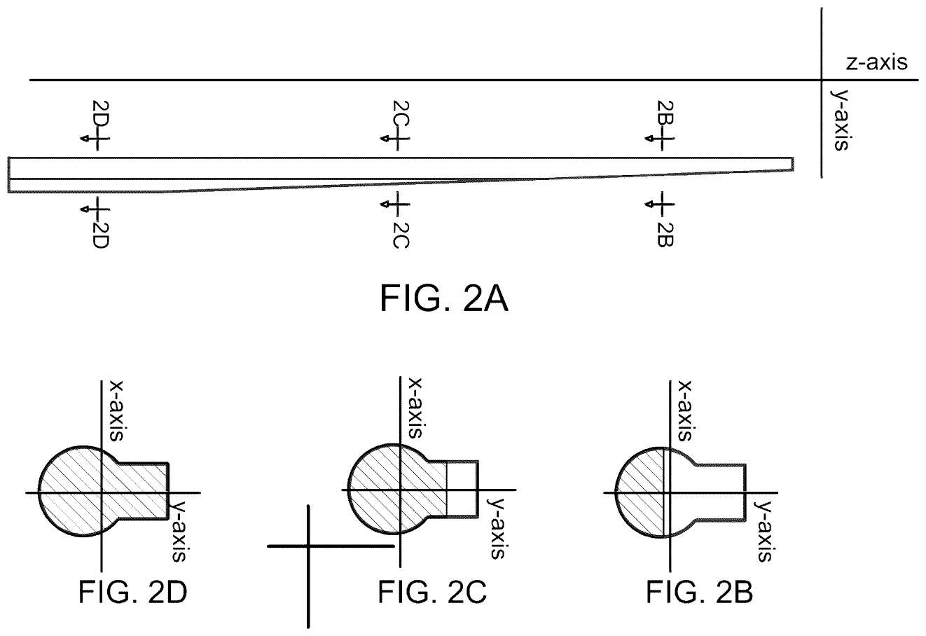

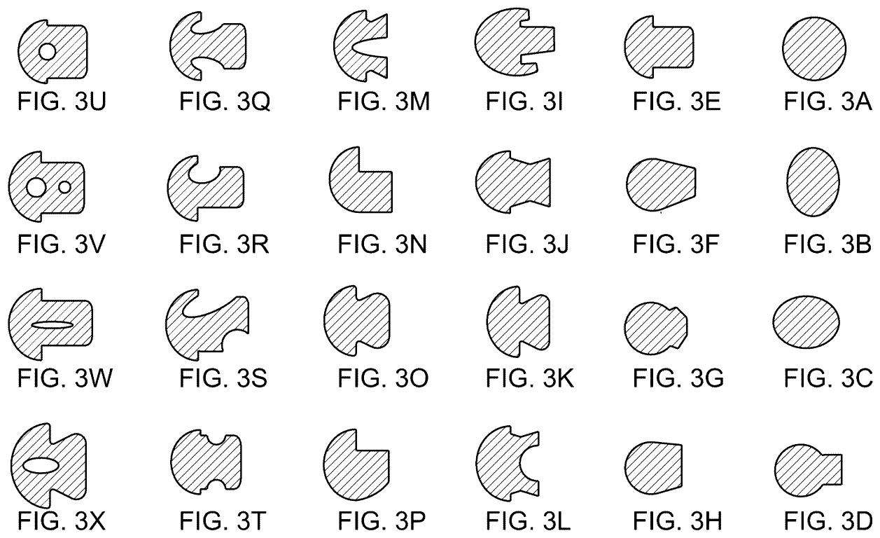

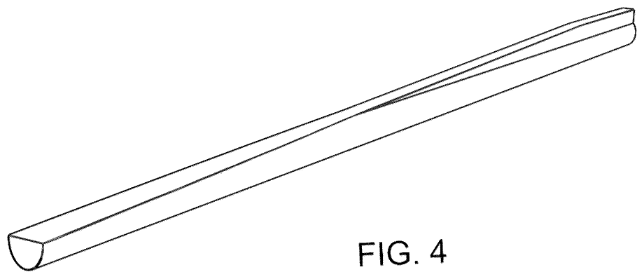

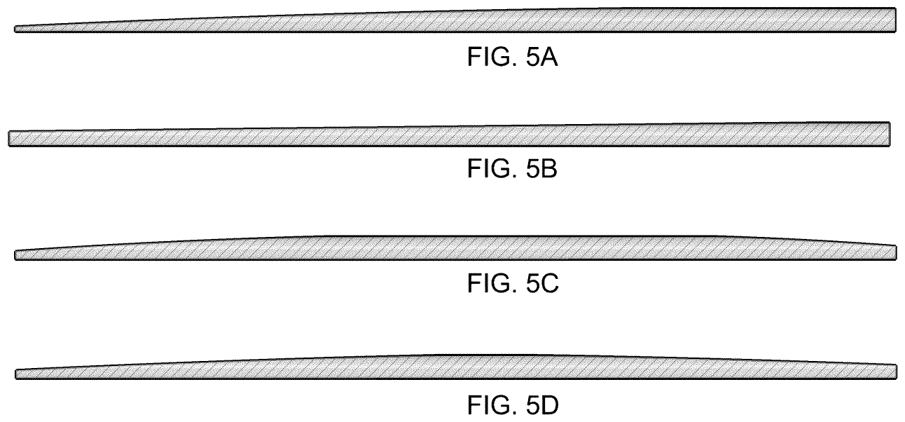

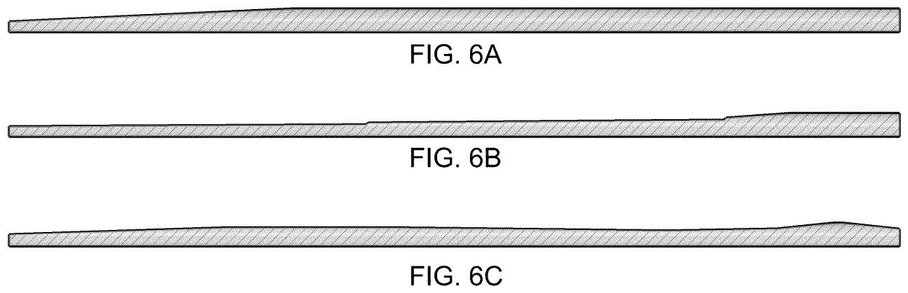

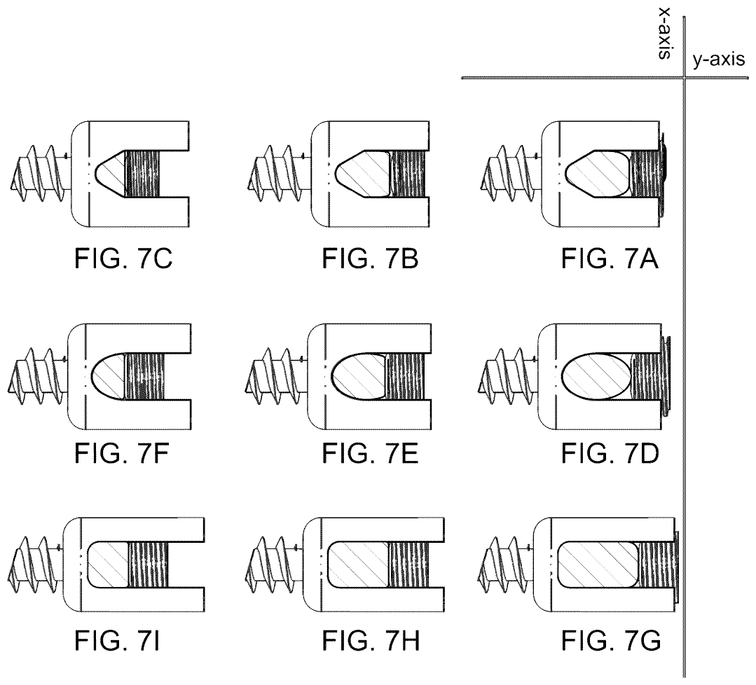

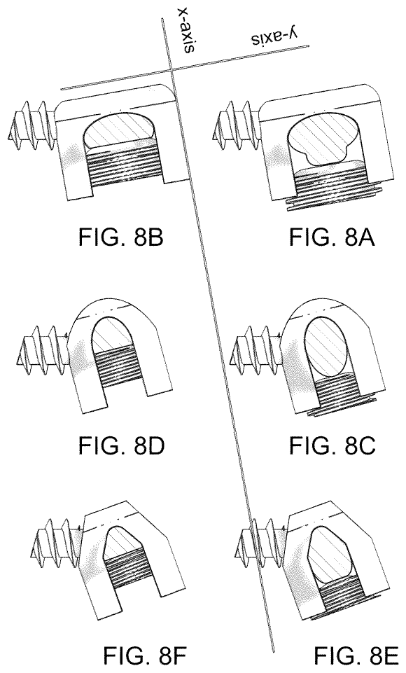

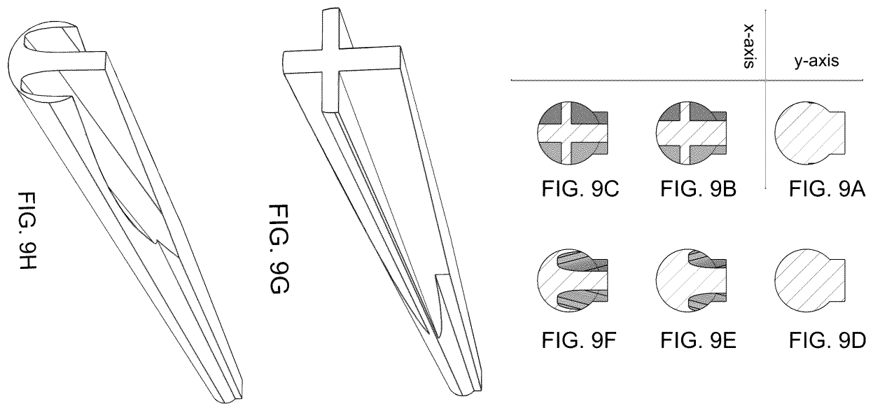

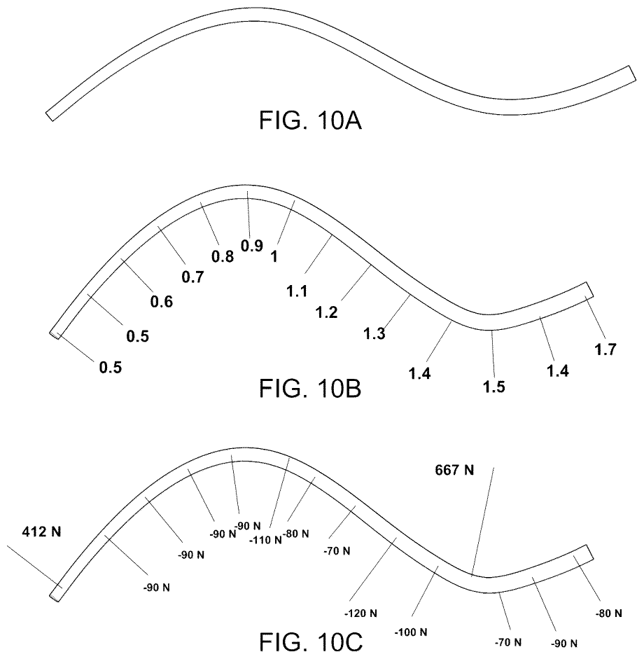

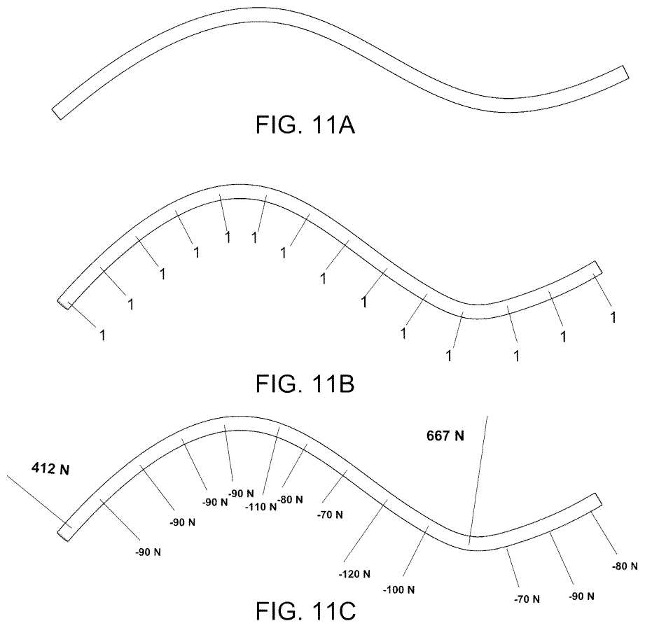

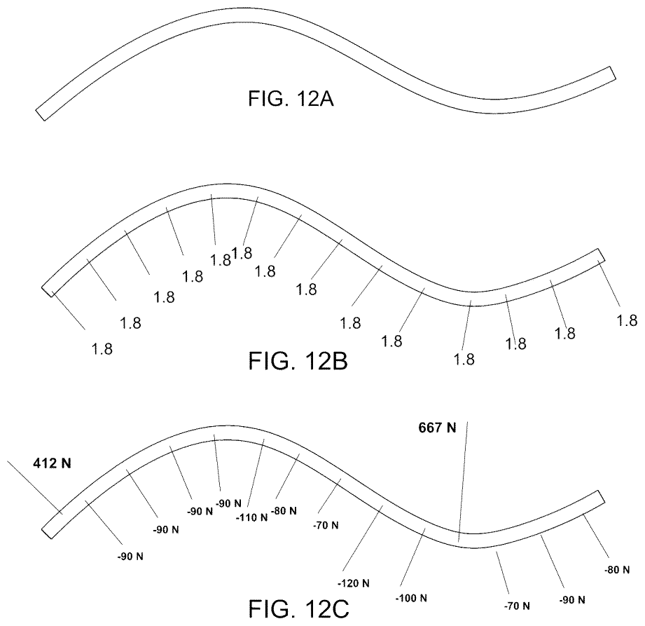

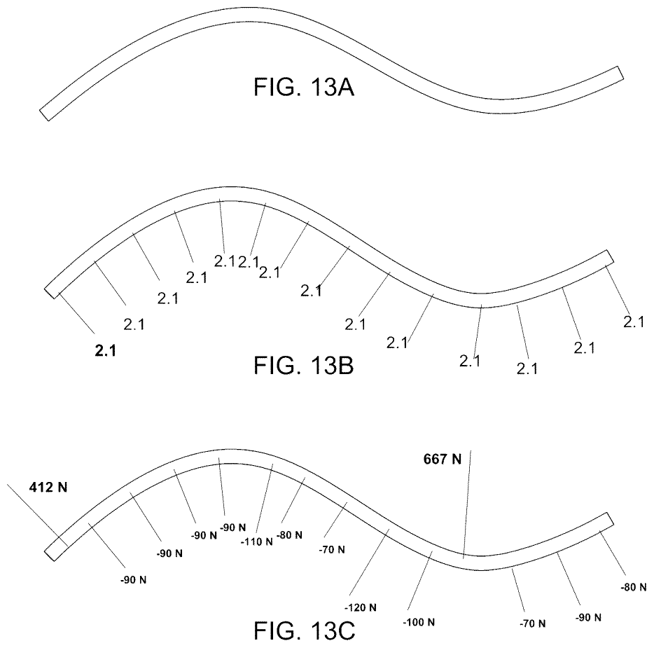

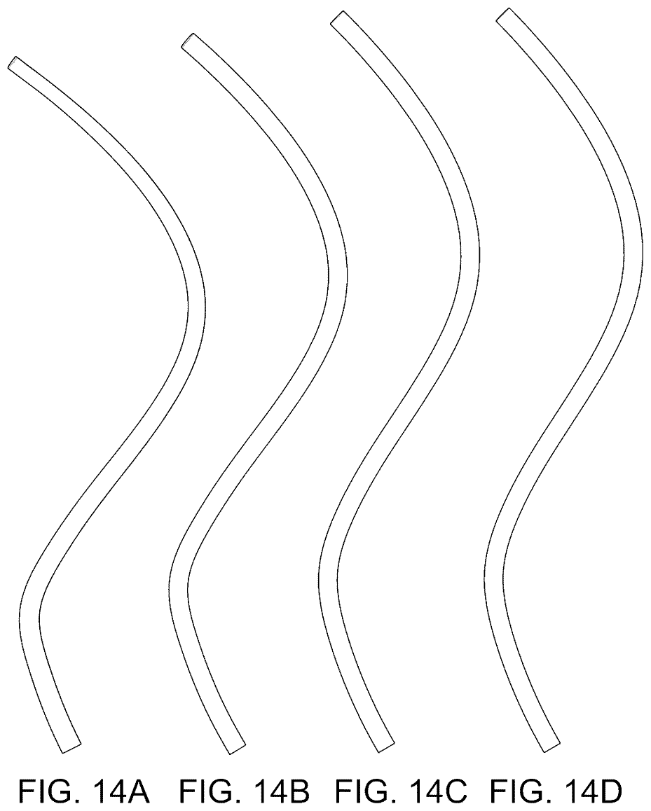

### METHOD OR DESIGNING AND FABRICATING THE NOVEL VMP SPINAL FIXING ROD

### Use of the Invention

The use of a particular embodiment of a VMP spinal fixing rod pertaining to this invention will be described in the context of correcting a spine deformity. However, this explanation is not intended to limit the applications of the new elements disclosed in this invention that can be utilized in any spine surgery where one or more vertebral elements need to be fixed or corrected and fixed in a definitive position using VMP spinal fixing rods designed to provide variable mechanical support and properties to the vertebral column, in a similar way to the design principles and fabrication techniques disclosed in this patent.

In a typical scenario, a patient with a spine deformity may require surgical intervention to correct one or more segments of the vertebral column. A primary goal of the procedure will be to regain a balanced alignment of the spine, which will involve surgically correcting the vertebral column deformity and fixing the spine in the corrected position with the help of one or more VMP spinal fixing rods. In some cases, the vertebral column fixation may extend proximally to include the cranium and/or distally to include the sacrum-pelvis unit.

The surgeon or caretaker, using dedicated medical imaging and surgical planning software described later in this document, will plan the desired three-dimensional final contour of the corrected spine before surgery. The surgeon will evaluate or estimate the desired or most convenient mechanical requirements for the different segments or levels of the VMP spinal fixing rod(s) that will be used to definitively fix the vertebral column in its desired final state. These mechanical requirements relate not only to the forces needed to correct the spine from its malaligned or deformed initial state to its final intraoperative corrected state but also to the mechanical forces that the spine and the VMP spinal fixing rods will sustain physiologically after surgery and during the patient's normal daily activities.

Although the precise mechanical requirements of the VMP spinal fixing rod(s) to be used and implanted in a particular patient, to fix the spine in its initial state or to correct and fix the spine in a definitively corrected position, cannot be precisely known with today's methods and technologies, these will vary depending on the patient's height, weight, general status, type of deformity, amount of correction needed, and the surgical procedure to be performed. Based on these parameters and the surgeon's experience and knowledge, an approximation of the desired flexural rigidity and resistance to fatigue that the VMP spinal fixing rod(s) will need along the length of the different vertebral segments or levels of the spine to be surgically fixed will be made.

### Design and Planning of a VMP Spinal Fixing Rod

In a preferred surgical planning process, the surgeon will assign “relative values” for the desired mechanical properties of the longitudinal VMP spinal fixing rod(s) to be used. These relative values will include parameters such as composition material, transverse cross-sectional shape and/or area, and bending stiffness along the different axes (x, y, z) of the VMP spinal fixing rod. The relative values of these and other mechanical parameters, if so desired, will be expressed as ratios compared to the properties of one or more known “standard” cylindrical rods of a specific diameter and composition, which the surgeon currently uses and is familiar with. This comparison will facilitate the analysis of the rigidity and fatigue resistance of the VMP spinal fixing rod(s), utilizing, for example, the cross-sectional geometric values and second area moments along the different axes of the transverse cross-section.

For example, the surgeon may determine that a particular VMP spinal fixing rod will need to have twice the bending stiffness of a circular cross-section rod of a specific diameter and composition at one segment, 1.5 times the bending stiffness at another segment, and 0.5 times its bending stiffness elsewhere along the length of the new VMP spinal rod to be designed. These relative values (2, 1.5, 0.5) are provided as examples, but any other relative value and number of segments or levels along the major length of the VMP spinal fixing rod can be chosen at the surgeon's discretion where appropriate or desirable.

As an initial step in planning and designing a VMP spinal fixing rod, the surgeon will have the option to select one or more parameters that will guide the design of the rod. These parameters may include, among others:
Composition
Diameter
Transverse cross-section shape
Maximum height
Maximum bending stiffness
Minimum bending stiffness

As is obvious to those skilled in the art, the selection of materials for the composition and fabrication of the rod, its diameter, transverse cross-section shape, maximum and minimum bending stiffness, and any other parameters related to the design of a VMP spinal fixing rod, can be limited by the technological constraints of the current state of the art. Additionally, the spatial and morphological constraints of the other fixing elements to which the VMP spinal fixing rod will be attached, as well as the anatomical particularities of the patient to be surgically treated, must be considered. Therefore, the chosen materials, diameters, and other expected mechanical properties of the VMP spinal fixing rod must align with what is feasible to fabricate and surgically implant at any given time.

In a preferred embodiment, the change in the cross-sectional area of the rod(s) to be designed and fabricated will be continuous and smooth. However, steeper and even discontinuous transitions in the cross-sectional area between different segments or levels of the longitudinal VMP spinal fixing rod can also be chosen.

As an example, the surgeon may opt for a VMP spinal fixing rod with a “closed U-shape” transverse cross-section of a particular constant width (diameter). The selected transverse cross-section shape, diameter, and composition of the VMP spinal fixing rod must be suitable for the proximal housing of the vertebral fixing elements attached to the vertebrae along the entire length of the spinal fixation, facilitating easy insertion of the VMP spinal fixing rod while also ensuring close contact fixation of the VMP spinal fixing rod within the vertebral fixing elements.

### Fabrication and Implementation

When a VMP spinal fixing rod with the specified material, diameter, and variable mechanical properties along its major length has been selected and accepted by the medical imaging and surgical planning software, it can then be sent for fabrication. As is obvious to those skilled in this art, a unique and unequivocal method of identifying the patient in whom the VMP spinal fixing rod(s) will be eventually implanted must accompany the order to fabricate the specified custom-made VMP spinal fixing rod(s). Many forms of personal and unique identification can be devised to be used with the VMP spinal fixing rod(s) as described in this invention, including but not limited to: the patient's name or identification, the surgeon ordering the rod, the institution/hospital where the VMP spinal fixing rod is to be implanted, the type of surgical procedure, the vertebral levels where the VMP spinal fixing rod is to be implanted, composition, and the mechanical and geometric data of the VMP spinal fixing rod (e.g., width, height).

With all the aforementioned specifications for the design of a VMP spinal fixing rod(s), the surgeon will have the choice to order a straight, non-contoured rod of a predetermined and desired length or to plan for a VMP spinal fixing rod that will be contoured to be adapted to the patient's spine in the desired final or corrected shape. If a custom-made contour of the VMP spinal fixing rod has not been designed, the VMP spinal fixing rod can be delivered in a straight form or in a predetermined “out of the box” contoured form, typically in a “C” or “S” shape. These VMP spinal fixing rods, which do not have a contour specifically designed by the surgeon, can be shaped by the surgeon, if so desired, during the surgical procedure or before the surgical procedure and then sterilized.

### Virtual Planning

With the help of dedicated medical imaging and surgical preplanning software, the surgeon may in some cases want to use a VMP spinal fixing rod of a specific width, material, transverse cross-section shape, and/or maximum and minimum dimensions. However, given certain pre-established mechanical requirements planned before surgery, it may not be possible or advisable to fabricate such a rod due to dimension constraints or steep transitions between segments with different mechanical requirements that could preclude optimal use in a surgical scenario. In this case, the software will offer the surgeon other virtual options for the VMP spinal fixing rod design that closely approximate the predetermined mechanical requirements and other physical characteristics initially requested, such as width, material, transverse cross-section shape, and maximum and minimum depth. The surgeon can then decide on the most appropriate rod design to be fabricated if so desired.

In a preferred embodiment, using specific and dedicated medical imaging and surgical preplanning software, images of the patient's spine will be loaded to visualize both the initial presurgical state and the final corrected position where the surgeon plans to fix the spine with VMP spinal fixing rods. Virtual images of longitudinal VMP spinal fixing rods along the desired segment(s) of the spine will be displayed and measured in length. The visualization of the rod(s) over the patient's medical images can be shown, if so desired, in both the virtually corrected vertebral column and the patient's spine in its initial presurgical condition, but in a preferred embodiment, at least the rod(s) will be shown over the virtually corrected images of the patient's spine. The medical images of the patient's spine, when appropriately treated using the tools in the medical imaging and surgical preplanning software, will allow the surgeon to visualize the patient's vertebral column anatomy and/or VMP spinal fixing rod(s) in a three-dimensional projection. This three-dimensional visualization is a software reconstruction of volumetric medical images such as a CT-scan or MRI-scan of the patient's vertebral column, or a virtual renderization of the vertebral column based on two-dimensional images of the patient's spine like radiographs.

In one preferred embodiment, using specific and dedicated medical imaging and surgical preplanning software, the surgeon will decide on the desired mechanical properties of the rod(s) for use during surgery and for the definitive fixation of the spine. The mechanical requirements of the longitudinal VMP spinal fixing rod(s) can be the same or different for the various rods planned for implantation, with variable mechanical strength along the entire length of each rod if so desired and as planned before surgery.

In one embodiment, the specific dimensions and the transverse cross-section area of the VMP spinal fixing rod(s) will be shown, if so desired, along one or more segments of the rod(s), or its entire length, precisely illustrating the mechanical properties of the rod(s) to the surgeon. In an ideal embodiment of such dedicated medical imaging analysis and surgical planning software, one or more variables of the rod's mechanical properties, geometry, and dimensions can be selected to be displayed separately or simultaneously. In another embodiment of such medical imaging and surgical planning software, many other alternative mechanical, material, and geometric parameters and values of the VMP spinal fixing rod(s) can be displayed, among which can be included the yield stress, maximum supported stress, buttress stress, and flexural rigidity in the sagittal, coronal, and axial planes.

As it may be difficult for surgeons to calculate and decide beforehand the precise mechanical requirements of the VMP spinal fixing rod(s) to be used in a particular patient that needs surgery, in one preferred embodiment, these mechanical properties of the VMP spinal fixing rod(s) will be represented in the medical imaging and surgical planning software, not as absolute values but as ratio relationships. These ratio relationships will be related to the mechanical properties of one or more conventionally used rods with which the surgeon is familiar and wants to compare the new VMP spinal fixing rod(s) to be designed. This relative specification and representation of the mechanical requirements of the VMP spinal fixing rod(s) to be designed will permit the surgeon to express the values of flexural rigidity and/or maximum stress loads of the rod(s) as a ratio between the rod(s) to be designed and these other known rod(s). For instance, the surgeon might design a VMP spinal fixing rod having three times the flexural rigidity in the sagittal plane of a 6.35 mm conventional chrome cobalt rod in the most distal-caudal ending of the rod, while having only half the flexural rigidity of a 5.5 mm titanium rod at its most proximal-cranial end. In a preferred embodiment, the surgeon can specify as many mechanical values and/or geometric values or constraints along the entire rod's length as desired in a similar manner.

In a preferred embodiment, while planning the mechanical requirements of a particular rod(s) with the dedicated medical imaging and surgical planning software, the surgeon can specify the particular mechanical properties of the VMP spinal fixing rod(s) using variables like maximum strength, flexural rigidity in the sagittal plane, flexural rigidity in the coronal plane, or flexural rigidity in the axial plane, among others. As it will be apparent to those skilled in this art, not all these mechanical properties may be simultaneously compatible for all the possible values that the surgeon may want to assign to these parameters along the length of the rod(s) to be designed, given other possible constraints imposed on the rod(s) such as composition material, width, height, and/or cross-section. In a situation where a VMP spinal fixing rod(s) with the specifications proposed by the surgeon is not possible or advisable to fabricate, the dedicated medical imaging and surgical planning software will offer one or more “compromise solutions” for the VMP spinal fixing rod(s) design that may closely satisfy one or more of the specified mechanical requirements, making appropriate suggestions to the surgeon in terms of material composition of the rod(s), maximum and/or minimum geometric dimensions, and/or shape of the transverse cross-section.

A more precise adjustment of the desired mechanical properties of a custom-made longitudinal fixing rod can be achieved with the dedicated medical imaging and surgical planning software if no prior choice of transverse cross-section shape, width, maximum height, minimum height, or composition is given. In this situation, the dedicated medical imaging and surgical planning software will offer one or more “compromise solutions” for the design of the VMP spinal fixing rod(s) that may more closely or completely satisfy the specified mechanical requirements.

Detailed Workflow for the Employment of Custom-Made Designed VMP Spinal Fixing Rods as Depicted in this Invention

Below is a comprehensive workflow necessary when performing a surgical intervention on a patient requiring the use of one or more VMP spinal fixing rods as described in this invention. In such surgical procedures, whether these VMP spinal fixing rod(s) to be employed have the same or variable mechanical properties along their major length, certain steps should be common and essential to all these surgeries. These include the previous evaluation and identification of a patient needing spine surgery and the assessment of one or more medical images of the patient in varying degrees of detail.

Additionally, planning for the surgical implants to be used, including the type of VMP spinal fixing rods, their diameter, and material composition, is crucial. However, the surgeon may decide not to engage deeply in designing the precise correction and/or fixation of the spine, nor determine or evaluate the specific mechanical requirements of the spine and the VMP spinal fixing rods to be used. Instead, the surgeon may choose to order “off-the-shelf” VMP spinal fixing rods that already provide variable mechanical properties along their length as described in this invention.

These “off-the-shelf” VMP spinal fixing rods can be provided as straight or pre-bent. The pre-bending can be adapted to the contour specified by the surgeon or to predetermined contours provided by the VMP spinal fixing rod manufacturer. All rods provided in this manner can be further recontoured by the surgeon if needed or desired, either before surgery or during the surgical procedure.

Alternatively, the surgeon can order off-the-shelf straight or pre-bent rods with already predetermined mechanical properties along their length as pre-established by the manufacturer-provider of the rod(s) following the same principles of variable mechanical properties of the VMP spinal fixing rod as described in this invention, without requiring exhaustive preoperative analysis and planning of the precise contour of the corrected vertebral column or the particular mechanical requirements of the rods for a particular patient.

All these rods should be considered under the same scope of this invention when serving the same purpose of providing predetermined and different mechanical properties along the rod's length when they have been constructed following the guidelines depicted in this invention.

### Flowchart for Designing a VMP Spinal Fixing Rod

### 1. Patient Consultation and Diagnosis

Identification: Patient with a spine pathology and/or spine deformity.
Surgical Need: Assessment for potential use of spinal fixing rods and VMP spinal fixing rods.

### 2. Initial Surgical Evaluation

Medical Imaging Review: Surgeon evaluates the patient's X-rays, CT/MRI scans.
Initial Assessment: Determine the vertebral segments needing correction and/or fixation.

### 3. Medical Imaging and Surgical Preplanning Software

Determining Desired Custom Shape of Corrected/Fixed Spine:
   1. Final Contour Planning: Surgeon plans the desired final three-dimensional contour of the corrected spine.
   2. Force Evaluation: Estimation of forces needed for correction and those to be sustained post-surgery in the yz (sagittal), xz (corona) and xy (axial) planes.
Selecting Initial Parameters of VMP Spinal Fixing Rod:
   1. Parameters Selection:
      Material composition
      Initial transverse cross-section shape
      Diameter (width, height)
      Maximum height/width
      Minimum height/width
      Maximum angle of tapering
      Alternative transitions for segments requiring different mechanical properties
Assigning Relative Values of VMP Spinal Fixing Rod Mechanical Properties:
   1. Mechanical Property Values: Mechanical resistance to fatigue and bending stiffness along x, y, and z axes along the length of the rod. Values are expressed as real physical nominal magnitudes or ratios compared to known standard spinal fixing rods.
Simulation of Medical Imaging and VMP Spinal Fixing Rods:
   1. Visualization: Display initial and final corrected positions of the spine.
   2. Virtual Spinal Fixing Rods: Display virtual images of spinal fixing rods.
Detailed Planning and Adjustment:
   1. Alternative Design Options: Software suggests alternative designs considering:
      Material composition
      Initial transverse cross-section shape
      Diameter (maximum width, maximum height)
      Alternative transitions for segments requiring different mechanical properties
   2. Compromise Solutions: Software offers solutions for fabrication based on surgeon planning requirements, physical constraints of VMP spinal fixing rods, and compatibility with vertebral fixing elements.
   3. Final Decision: Surgeon decides on the final material composition, initial transverse cross-section shape, diameter, and overall mechanical properties from the available options.

### 4. Order Custom VMP Spinal Fixing Rod Fabrication

Design Verification: Surgeon verifies and accepts the VMP spinal fixing rod design in the software.
Patient Identification: Attach unique patient identification information to the order.
Order Placement: Send order for custom-made VMP spinal fixing rods fabrication.

### 6. Manufacturing Process

Material Selection: Based on surgical preplan specifications.
Fabrication: Using CNC machining, laser cutting, or 3D printing to create the custom-shaped VMP spinal fixing rod with planned variable height/width.
Molding VMP Spinal Fixing Rod to Three-Dimensional Shape: If the VMP spinal fixing rod is fabricated as a straight rod, it will be contoured to the required three-dimensional shape.
Heat Treatment: Improve molecular structure and enhance mechanical properties like fatigue resistance and strength if the VMP spinal fixing rod has been mechanically bent.
Labeling and Identification: Laser engraving or other methods to mark VMP spinal fixing rods with unique identifiers for traceability and identification.
Sterilization: Ensure VMP spinal fixing rods are free of contaminants before surgical use.
Packaging: Package sterilized VMP spinal fixing rods in sterile, secure containers.

### 6. Delivery/Shipment to the Surgeon

Shipment: Ship final VMP spinal fixing rods to the healthcare facility or surgeon's office, maintaining integrity and sterility.

### 7. Surgical Implementation

Pre-Surgery Review: Surgeon reviews custom-made VMP spinal fixing rods to ensure they match the pre-surgical plan, making any necessary adjustments.
Intraoperative Adjustments: Custom-made VMP spinal fixing rods can be reshaped or cut during the surgical procedure if needed.

The detailed above workflow in the planning of customized VMP spinal fixing rod(s) with variable mechanical properties serves as an example of a general procedure where a surgeon or caretaker aims to use one or more VMP longitudinal spinal fixing rods providing different mechanical properties along the rod's length, if so desired or needed. The workflow comprises a complete set of actions for the surgeon to design a VMP spinal fixing rod satisfying most or all of the desired mechanical properties selected before surgery for a particular patient.

However, this particular description of a possible workflow using the elements and processes described in this invention does not imply that a surgeon or caretaker treating a patient with a spine pathology requiring one or more VMP spinal fixing rods must follow each and every step in the specified order to obtain a rod with at least two differentiated mechanical properties along its length as described in this invention. The steps described above—medical imaging analysis, defining the particular or desired contour of a VMP spinal fixing rod, and determining its material composition, length, mechanical properties, and geometric characteristics—can alternatively be performed using a combination of one or more different software tools or packages. Various medical imaging, graphic design, and mechanical engineering software packages, or a combination thereof, can be used to design a VMP spinal fixing rod with the same variable mechanical properties as described in this invention. These alternative methods and tools are considered within the scope of this invention when they have the same intention of selecting or designing and fabricating a VMP spinal fixing rod as disclosed in this invention.

### Flowchart of Alternative Ways for the Use of VMP Spinal Fixing Rods

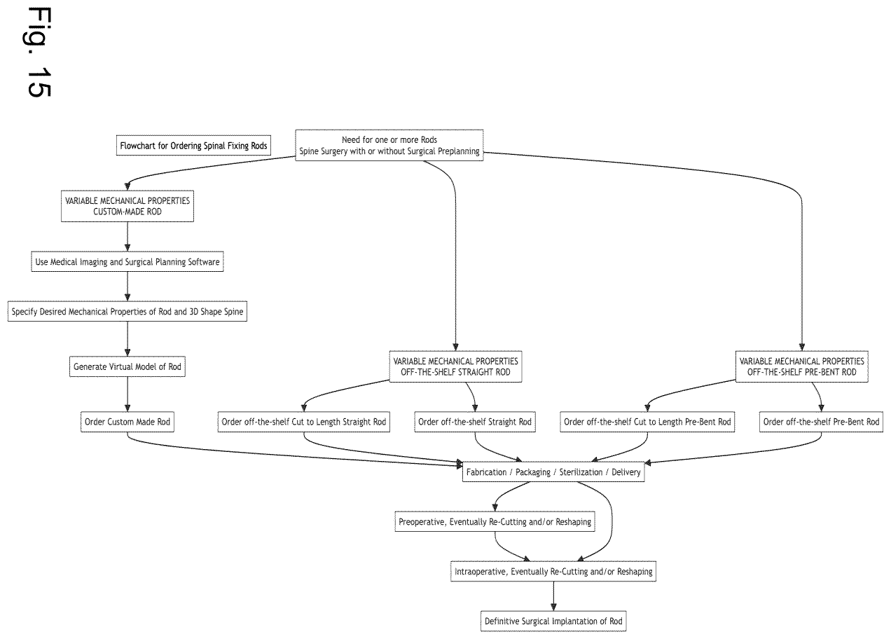

For clarity and simplification, in the following flowchart seen in FIG. 15, “rod” refers to a VMP spinal fixing rod described in this invention.

Having now described a few embodiments of the invention, it should be apparent to those skilled in the art that the foregoing is merely illustrative and not limiting, having been presented by way of example only. Numerous modifications and other embodiments are within the scope of one of ordinary skill in the art and are contemplated as falling within the scope of the invention and any equivalents thereto. Variations of the present invention would be readily apparent to those skilled in the art, and the present invention is intended to include those alternatives. Furthermore, since numerous modifications will readily occur to those skilled in the art, it is not desired to limit the invention to the exact construction and operation illustrated and described. Accordingly, all suitable modifications and equivalents may be resorted to, falling within the scope of the invention.

For clarity, references to the x-axis, y-axis, and z-axis in this application should be understood to correspond to the width, height, and longitudinal direction of the spinal fixing rod's transverse cross-section and length, respectively, regardless of nomenclature used in any competing device. Such definitions apply consistently to all claims and drawings herein.

Clarification: As used in the present claims, the term “spinal fixing rod” shall be understood to include rods having variable mechanical properties (VMP), as described throughout the specification. Accordingly, the features, configurations, and variations attributed to ‘VMP spinal fixing rods’ in the specification shall be deemed to be included in and apply to any reference to ‘spinal fixing rod’ within the claim.

1. A spinal fixing rod for surgical fixation or correction of one or more vertebrae, comprising:
(a) an elongated rod body having a longitudinal axis (z-axis) and a transverse cross-sectional shape selected from the group consisting of circular, elliptical, polygonal, and combinations thereof, wherein the x-axis corresponds to the width of the transverse cross-section, and the y-axis corresponds to the height of the transverse cross-section;
(b) a differentiated transverse cross-sectional profile along at least a portion of the longitudinal axis, configured to provide different mechanical properties along the length of the rod; and
(c) wherein the rod is formed of a biocompatible material suitable for implantation, and changes in geometry along the x-axis and y-axis contribute to variations in mechanical properties along the z-axis and to increased or decreased rotational rigidity about the z-axis.

2. The spinal fixing rod of claim 1, wherein the transverse cross-sectional profile varies by:
(a) a reduction in height along one or more segments while maintaining width; or
(b) a modification of the perimeter geometry of the transverse cross-section, wherein material is selectively removed primarily along the y-axis while maintaining the maximum height, such that the second moment of area is reduced and bending resistance is decreased predominantly along the x-axis.

3. The spinal fixing rod of claim 1, wherein the transverse cross-sectional profile varies by:
(a) a reduction in width along one or more segments while maintaining height; or
(b) a modification of the perimeter geometry of the transverse cross-section, wherein material is selectively removed primarily along the x-axis while maintaining the maximum height, such that the second moment of area is reduced and bending resistance is decreased predominantly along the y-axis.

4. The spinal fixing rod of claim 1, wherein the transverse cross-sectional profile varies by a modification of the perimeter geometry, wherein material can be selectively removed along either or both the x-axis and y-axis, while maintaining the maximum width and height of the cross-section, such that the second moment of area is reduced and bending resistance is decreased along both the x-axis and y-axis.

5. The spinal fixing rod of claim 1, wherein the rod includes at least one hollowed portion along its length, the hollowed portion having a differentiated transverse cross-sectional profile, either in external contour or internal void geometry, along at least a portion of the longitudinal axis, configured to provide different mechanical properties along the rod.

6. The spinal fixing rod of claim 1, wherein the overall length and mechanical property variations are pre-established by the manufacturer, and wherein the rod is provided in a form selected from the group consisting of:
(a) a straight configuration;
(b) a pre-bent standard curvature configuration; and
(c) a custom-molded configuration based on surgeon preferences or patient-specific needs.

7. The spinal fixing rod of claim 1, wherein the variation in mechanical properties is determined based on preoperative surgical planning according to surgeon preferences and/or patient-specific requirements.

8. A method for customizing a spinal fixing rod for use in spinal surgery, comprising:
(a) obtaining patient-specific medical imaging data;
(b) determining mechanical requirements along one or more spinal segments;
(c) generating a rod design having a variable transverse cross-sectional profile corresponding to the mechanical requirements; and
(d) fabricating the rod from a biocompatible material based on the generated design.

9. The method of claim 8, wherein the rod is provided either in:
(a) a straight configuration to be contoured intraoperatively by the surgeon; or
(b) a standard bent configuration to be further contoured intraoperatively.

10. The method of claim 8, further comprising custom contouring the rod to a desired three-dimensional shape based on:
(a) the final planned spinal anatomy after surgery; or
(b) a desired final configuration of the rod and spine after expected intraoperative or postoperative deformation resulting from surgical correction or physiological loading.

11. A computer-implemented system for planning and designing custom spinal fixing rods, comprising:
(a) a user interface configured to receive medical imaging data of a patient's spine;
(b) a planning module configured to define mechanical requirements along one or more spinal segments;
(c) a design engine configured to generate a spinal fixing rod profile having variable transverse cross-sections based on said mechanical requirements; and
(d) an output module configured to provide a rod fabrication specification.

12. The system of claim 11, further comprising a simulation module configured to model rod deformation under expected loads.

13. The system of claim 11, wherein the mechanical requirements can be expressed as relative ratios with respect to one or more standard spinal rods.

14. The system of claim 11, wherein the user interface enables specification of maximum and minimum dimensions for rod width and height.

15. The system of claim 11, wherein the output module provides feedback on design feasibility and suggests alternative configurations when user-specified mechanical requirements render fabrication of the spinal rod either impossible or when user-specified mechanical requirements render fabrication of the spinal rod either technically infeasible, unsafe, or otherwise not recommended based on system-defined constraints.
